(19) United States
(12) Patent Application Publication
(10) Pub. No.: US 2023/0166013 A1
(43) Pub. Date: Jun. 1, 2023

(54) ANTITHROMBOTIC CELL ADHESION SHEET AND MEDICAL DEVICE WITH SHEET

(71) Applicants: FUJIFILM Corporation, Tokyo (JP); KYUSHU UNIVERSITY, NATIONAL UNIVERSITY CORPORATION, Fukuoka (JP)

(72) Inventors: Atsushi SUGASAKI, Kanagawa (JP); Yosuke Yamamoto, Kanagawa (JP); Yuta Shigenoi, Kanagawa (JP); Masaru Tanaka, Fukuoka (JP); Shingo Kobayashi, Fukuoka (JP)

(73) Assignees: FUJIFILM Corporation, Tokyo (JP); KYUSHU UNIVERSITY, NATIONAL UNIVERSITY CORPORATION, Fukuoka (JP)

(21) Appl. No.: 18/154,039

(22) Filed: Jan. 13, 2023

Related U.S. Application Data
(63) Continuation of application No. PCT/JP2021/027098, filed on Jul. 20, 2021.

(30) Foreign Application Priority Data
Jul. 22, 2020 (JP) 2020-125538

(51) Int. Cl. A61L 33/06; A61L 31/04; A61L 29/04; A61L 27/16

(52) U.S. Cl. CPC A61L 33/064; A61L 31/048; A61L 29/041; A61L 27/16

(57) ABSTRACT

A first object of the present invention is to provide an antithrombotic cell adhesion sheet having excellent antithrombotic properties and cell adhesion properties. In addition, a second object of the present invention is to provide a medical device with a sheet using the antithrombotic cell adhesion sheet.

The antithrombotic cell adhesion sheet of the present invention is a sheet formed b using a compound represented by General Formula (1).

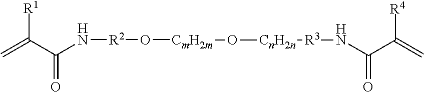

In General Formula (1), R¹ and R⁴ each independently represent a hydrogen atom or a methyl group. R² and R³ each independently represent —CH₂CH(R^{A1})CH₂—. p and r represent 1. q represents 0 or 1. m and n each independently represent an integer of 2 to 6. R^{A1} represents a hydrogen atom or a methyl group.

### CROSS-REFERENCE TO RELATED APPLICATIONS

This application is a Continuation of PCT International Application No. PCT/JP2021/027098 filed on Jul. 20, 2021, which claims priority under 35 U.S.C. § 119(a) to Japanese Patent Application No. 2020-125538 filed on Jul. 22, 2020. The above applications are hereby expressly incorporated by reference, in its entirety, into the present application.

### BACKGROUND OF THE INVENTION

### 1. Field of the Invention

The present invention relates to an antithrombotic cell adhesion sheet and a medical device with a sheet.

### 2. Description of the Related Art

In recent years, a separation and recovery technology such as a method for selectively separating and recovering a target substance such as a cell, a physiologically active substance, and a protein from a body fluid such as blood, a tissue fluid, and a lymph fluid, and a method for separating and removing a bacterium, a virus, or the like from a biological tissue fluid has been used for preventing an autoimmune disease, an acquired immunodeficiency syndrome, an acute rejection reaction after transplantation, and the like.

For cell medical care of detecting and treating cancer cells derived from a biological tissue that has become cancerous, including leukemia, which is a blood cancer, a technology for efficiently separating and recovering the cancerous cells with high sensitivity is important. In recent years, instead of biopsy in which cancer cells are directly collected from the cancerous tissue, blood biopsy (liquid biopsy) in which tumor markers or cancerous cells are detected from biological tissue fluid typified by blood has attracted attention. An examination by tissue collection performed in the related art is a highly invasive method for a subject, whereas a minimally invasive method called blood collection is used in blood biopsy. Thus, the feature is that the burden on the subject's body is extremely light. On the other hand, in the examination using tumor markers by blood biopsy, a site-specific tumor marker is rarely established in the cancerous tissue. Thus, there is a need for development of a technology for specifically capturing and detecting cancer cells (circulating cancer cells in the blood) that slightly leak into the blood from the cancerous tissue and circulate in the body with high sensitivity and high efficiency.

For example, JP2012-105579A describes “a hydrated composition for separating and collecting cancer cells in which the amount of intermediate water of 30 wt % or less”, as a hydrated composition used in a cell separation method for selectively adsorbing and separating predetermined cells present in a living body (Claim 4). Specifically, a hydrated composition for separating and collecting cancer cells including a polymer represented by the following Formula (1) is disclosed.

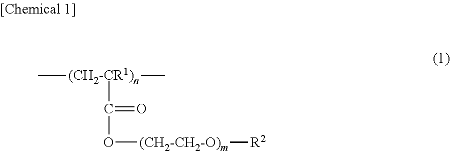

Incidentally, for example, medical devices that are introduced into a human body for a long period of time, such as an artificial blood vessel, a catheter, and a stent, are usually preferably made of a material that is compatible with a substance constituting a living body and is less likely to be contaminated. In particular, there is a strong demand that blood clots are less likely to be generated. Thus, attempts have been made to coat a surface of the medical device with the material described above. It is known that a compound having an oxyethylene repeating structure [—(O—C₂H₅—)n-] is effective as a material for suppressing the generation of blood clots (see, for example, Teruo Okano et al., “Biocompatible Polymers”, Kyoritsu Shuppan Co., Ltd. (1988), Hiroo Iwata, “Biomaterials”, Kyoritsu Shuppan Co., Ltd. (2005), and Toshihiro Akaike, “Biological Functional Materials—Basics of Artificial Organs, Tissue Engineering, and Regenerative Medicine—”, Corona (2005)).

### SUMMARY OF THE INVENTION

The present inventors have formed a film (sheet) by using a coating formulation containing a compound having an oxyethylene repeating structure, such as the hydrated composition for separating and collecting cancer cells disclosed in JP2012-105579A, and have examined film properties and have found that platelet adhesion was less likely to occur and had antithrombotic properties, but there was room for further improvement in cell adhesion properties (particularly tumor cell adhesion properties such as cancer cells). That is, the present inventors have found an object of further improving cell adhesion properties (particularly, tumor cell adhesion properties such as cancer cells) while having good antithrombotic properties.

Therefore, an object of the present invention is to provide an antithrombotic cell adhesion sheet having excellent antithrombotic properties and cell adhesion properties (particularly, tumor cell adhesion properties such as cancer cells).

Another object of the present invention is to provide a medical device with a sheet using the antithrombotic cell adhesion sheet.

The present inventors have found that the above-described objects can be solved by the following configurations.

[1] There is provided an antithrombotic cell adhesion sheet formed by using a compound represented by General Formula (1) to be described below.

[2] In the antithrombotic cell adhesion sheet according to [1], the q represents 1.

[3] In the antithrombotic cell adhesion sheet according to [1] or [2], the R^{A1} represents a hydrogen atom.

[4] In the antithrombotic cell adhesion sheet according to any one of [1] to [3], both the R¹ and the R⁴ represent a hydrogen atom.

[5] In the antithrombotic cell adhesion sheet according to any one of [1] to [4], both the m and the n represent 2.

[6] There is provided a medical device with a sheet comprising a medical device, and the antithrombotic cell adhesion sheet according to any one of [1] to [5] disposed on a surface of the medical device.

[7] In the medical device with a sheet according to [6], the medical device is selected from the group consisting of a stent, a graft, and a catheter.

[8] In the medical device with a sheet according to [6], a shape of the medical device is selected from the group consisting of a film, a sheet, a tube, a bag, a chalet, a dish, a well, a porous body, and a vial.

According to the present invention, it is possible to provide an antithrombotic cell adhesion sheet having excellent antithrombotic properties and cell adhesion properties (particularly, tumor cell adhesion properties such as cancer cells).

According to the present invention, it is possible to provide a medical device with a sheet using the antithrombotic cell adhesion sheet.

### DESCRIPTION OF THE PREFERRED EMBODIMENTS

Hereinafter, the present invention will be described in detail.

Although configuration requirements to be described below may be described based on a representative embodiment of the present invention, the present invention is not limited to such an embodiment.

In the present specification, a numerical range represented by using “˜” means a range including numerical values described before and after “˜” as an lower limit value and a upper limit value.

In the present specification, (meth)acrylamide means acrylamide and methacrylamide. (Meth)acrylate means acrylate and methacrylate. (Meth)acryloyl means acryloyl and methacryloyl.

In the present specification, a solid content is intended to be a component contained in a composition excluding a solvent component, and is regarded as a solid content even though properties thereof are liquid.

[Antithrombotic Cell Adhesion Sheet]

An antithrombotic cell adhesion sheet (hereinafter, abbreviated as a “sheet”) according to the embodiment of the present invention is a sheet formed by using a compound represented by General Formula (1) to be described below.

Platelets are less likely to adhere to the sheet according to the embodiment of the present invention having the above-described configuration, and the sheet has excellent cell adhesion properties. In particular, the above-mentioned sheet has excellent adhesion properties to tumor cells such as cancer cells, and in particular, has excellent adhesion properties to MCF-7 (human-derived benign breast tumor cell line).

Although the mechanism of action for exhibiting the above effect is not clear, the present inventors have considered that the mechanism is caused by a structure of a chain that connects two (meth)acrylamide groups specified in General Formula (1) in General Formula (1).

An example of a method for manufacturing a sheet according to the embodiment of the present invention is a method using a curable composition containing the compound represented by General Formula (1) to be described below.

Hereinafter, the compound represented by General Formula (1) will be described first, and then the curable composition will be described.

[Compound represented by General Formula (1)]

Hereinafter, the compound represented by General Formula (1) will be described in detail.

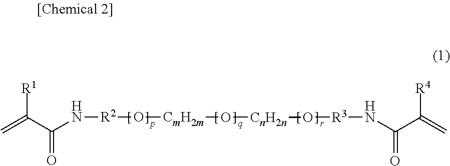

In General Formula (1), R¹ and R⁴ each independently represent a hydrogen atom or a methyl group.

R¹ and R⁴ may be the same or different from each other, but are preferably the same, and both R¹ and R⁴ are more preferably hydrogen atoms.

R² and R³ each independently represent —CH₂CH(R^{A1})CH₂—.

R^{A1} represents a hydrogen atom or a methyl group, and is preferably a hydrogen atom.

R² and R³ may be the same or different from each other, but are preferably the same.

p and r represent 1.

q represents 0 or 1, and is preferably 1.

m and n each independently represent an integer of 2 to 6, and preferably an integer of 2 to 4. In particular, m and n more preferably represent 2.

In General Formula (1), an alkylene group represented by CₘH₂ₘ and CₙH₂ₙ may be linear or branched, but is preferably linear.

m and n may be the same or different from each other, but are preferably the same.

In General Formula (1), in a case where q represents 0, a total number of carbons of the alkylene group represented by —CₘH₂ₘ—CₙH₂ₙ— may represent an integer of 4 to 12. Accordingly, General Formula (1) allows, for example, a case where m is 1 and n is 3, and a case where m is 3 and n is 1. The alkylene group represented by —CₘH₂ₘ—CₙH₂ₙ— may be linear or branched, but is preferably linear.

Hereinafter, specific examples of the compound represented by General Formula (1) will be shown, but the present invention is not limited thereto.

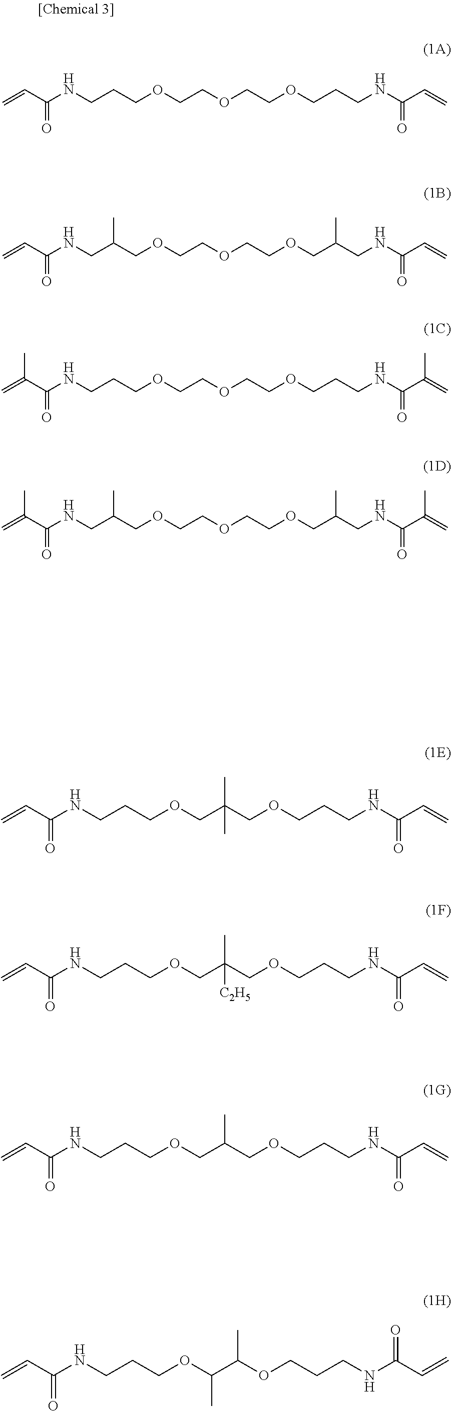

The compound represented by General Formula (1) can be synthesized by a known method in the related art.

[Curable Composition]

Hereinafter, each component contained in the curable composition will be described in detail.

The curable composition includes the compound represented by General Formula (1).

The compound represented by General Formula (1) is as described above.

For example, the curable composition contains, as a component other than the compound represented by General Formula (1), other polymerizable compound other than the compound represented by General Formula (1), a polymerization initiator, a polymerization inhibitor, or a solvent. In particular, the compound represented by General Formula (1), the polymerization initiator, and the solvent are preferably contained.

<Compound Represented by General Formula (1)>

The curable composition contains the compound represented by General Formula (1).

The compound represented by General Formula (1) may be used alone or in combination of two or more kinds.

In the curable composition, a content of the compound represented by General Formula (1) (in a case where a plurality of kinds are present, a total content thereof) is 50.0% by mass or more with respect to the total solid content of the composition, more preferably 75.0% by mass or more, still more preferably 85.0% by mass or more, and particularly preferably 95.0% by mass or more. An upper limit value is not particularly limited, but is, for example, 100% by mass or less, preferably 99.9% by mass or less, more preferably 99.5% by mass or less, still more preferably 98.0% by mass or less, and particularly preferably 97.5% by mass or less.

<Polymerization Initiator>

The curable composition may contain the polymerization initiator.

The polymerization initiator is not particularly limited, but is preferably a thermal polymerization initiator or a photopolymerization initiator, and more preferably a photopolymerization initiator.

Examples of the photopolymerization initiator include an alkynephenone-based photopolymerization initiator, a methoxyketone-based photopolymerization initiator, an acylphosphine oxide-based photopolymerization initiator, and a hydroxyketone-based photopolymerization initiator (for example, Omnirad 184; 1,2-α-hydroxyalkylphenone), aminoketone-based photopolymerization initiator (for example, 2-methyl-1-[4-(methylthio)phenyl]-2-morpholinopropane-1-one (Omnirad 907)), oxime-based photopolymerization initiator, and an oxyphenyl acetate ester-based photopolymerization initiator (Omnirad 754).

Examples of other initiators include azo-based polymerization initiators (for example, V-50 and V-601), persulfate-based polymerization initiators, peroxomonosulfate-based polymerization initiators, and redox-based polymerization initiators.

The polymerization initiator is also preferably a compound represented by the following (PI). The compound represented by the following (PI) can be used as a photopolymerization initiator.

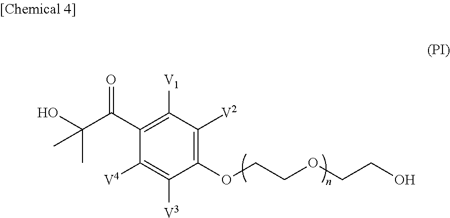

In General Formula (PI), V¹, V², V³, and V⁴ each independently represent a hydrogen atom or a substituent.

A kind of the substituent is not particularly limited, and examples thereof include a halogen atom, an alkyl group, an alkoxy group, a hydroxy group, an alkylthio group, a mercapto group, an acyl group, and an amino group.

The halogen atom is preferably a fluorine atom, a chlorine atom, a bromine atom, or an iodine atom, more preferably a chlorine atom or a bromine atom, and still more preferably a chlorine atom.

The number of carbons of the alkyl group and the alkoxy group is not particularly limited, but is preferably 1 to 6 and more preferably 1 to 3. The alkyl group and the alkoxy group may be linear, branched, or cyclic.

The number of carbons of the alkylthio group is not particularly limited, but is preferably 1 to 6 and more preferably 1 to 4. The alkylthio group may be linear, branched, or cyclic.

The number of carbons of the acyl group is not particularly limited, but is preferably 2 to 6 and more preferably 2 to 3. The acyl group may be linear or branched. Specific examples of the acyl group include a formyl group, an acetyl group, an ethyl acyl group, an n-propylacyl group, and an isopropylacyl group.

The amino group may be any one of a primary amino group (—NH₂), a secondary amino group (—NHR), or a tertiary amino group (—NR₂). Here, R represents a hydrocarbon group (for example, an alkyl group having 1 to 6 carbons). Two Rs in the tertiary amino group may be the same or different. Specific examples of the amino group include a dimethylamino group and a diethylamino group.

n represents an integer of 1 to 5, preferably an integer of 1 to 3, more preferably an integer of 1 to 2, and still more preferably 1.

The compound represented by General Formula (PI) is preferably a liquid under a condition of 23° C.

The compound represented by General Formula (PI) can be synthesized, for example, according to the methods described in paragraphs 0067 to 0071 and paragraphs 0112 to 0115 of JP2000-186242A.

Specific examples of the compound represented by General Formula (PI) include a compound represented by General Formula (VII) described in paragraphs 0064 to 0070 of WO2017/018146A.

In the curable composition, the polymerization initiator may be used alone or in combination of two or more kinds.

In a case where the curable composition contains the polymerization initiator, the content of the polymerization initiator (in a case where a plurality of kinds are present, a total content thereof) is 10.0% by mass or less with respect to the total solid content of the composition, more preferably 8.0% by mass or less, still more preferably 5.0% by mass or less, and particularly preferably 3.5% by mass or less. A lower limit thereof is preferably 0.1% by mass or more, more preferably 0.5% by mass or more, and still more preferably 1.0% by mass or more.

<Solvent>

The curable composition may contain a solvent.

A kind of the solvent is not particularly limited, and examples thereof include water and an organic solvent.

The organic solvent is preferably alcohols such as methanol and butanol, and ketones such as methyl ethyl ketone and methyl isobutyl ketone. In particular, the organic solvent is preferably alcohols having 3 or less carbons or ketones having 4 or less carbons, and more preferably methanol or acetone.

In the curable composition, the solvent may be alone or in combination of two or more kinds.

In a case where the curable composition contains the solvent, a content of a solid content of the curable composition is preferably 5.0% by mass or more with respect to a total mass of the composition, more preferably 10.0% by mass or more, and still more preferably 15.0% by mass or more. An upper limit value thereof is not particularly limited, but is preferably 90.0% by mass or less, more preferably 70.0% by mass or less, and still more preferably 50.0% by mass or less.

<Another Polymerizable Compound>

The curable composition may contain another polymerizable compound (hereinafter, also referred to as “another polymerizable compound”) other than the compound represented by General Formula (1) described above.

Another polymerizable compound is not particularly limited, but is preferably a compound having an ethylenically unsaturated group, and more preferably a compound having a (meth)acryloyl group or a (meth)acrylamide group. The number of ethylenically unsaturated groups in a molecule is not particularly limited, and is preferably, for example, 1 to 8, more preferably 1 to 4, and still more preferably 1 or 2.

In particular, another polymerizable compound is preferably a (meth)acrylate-based compound and a (meth)acrylamide-based compound.

Examples of the (meth)acrylate-based compound include methyl (meth)acrylate, ethyl (meth)acrylate, n-butyl (meth)acrylate, 2-ethylhexyl (meth)acrylate, 2-methoxyethyl (meth)acrylate, 2-hydroxyethyl (meth)acrylate, 2-hydroxy-1,3-propylene diol diacrylate, 2-hydroxypropyl (meth)acrylate, 3-(meth)acryloyloxypropylsulfonate, N,N-dimethylaminoethyl (meth)acrylate, N,N-diethylaminoethyl (meth)acrylate, polyalkylene glycol mono (meth)acrylate, polyalkylene glycol di(meth)acrylate, 2-(meth)acryloyloxyethyl methyl sulfoxide, tetraethylene glycol dimethacrylate, urethane dimethacrylate, and trimethylolpropanetri (meth)acrylate.

Examples of the (meth)acrylamide-based compound include (meth)acrylamide, N-methyl (meth)acrylamide, and N-2-hydroxyethyl (meth)acrylamide.

Another polymerizable compound may be used alone or in combination of two or more kinds.

In a case where the curable composition contains another polymerizable compound, a content of another polymerizable compound (in a case where a plurality of kinds are present, a total content thereof) is 80.0% by mass with respect to the total solid content of the composition, more preferably 50.0% by mass or less, and still more preferably 30.0% by mass or less. A lower limit thereof is preferably 0.1% by mass or more.

<Polymerization Inhibitor>

The curable composition may contain a polymerization inhibitor.

The polymerization inhibitor is not particularly limited, and examples thereof include 4-hydroxy-2,2,6,6-tetramethylpiperidin-1-oxyl (40H-TEMPO).

The polymerization inhibitor may be used alone or in combination of two or more kinds.

In a case where the curable composition contains the polymerization inhibitor, a content of the polymerization inhibitor (in a case where a plurality of kinds are present, a total content thereof) is preferably 0.0005 to 1% by mass with respect to the total solid content of the composition.

<Other Components>

The curable composition may contain components other than the above-described components. Examples of such a component include a polyfunctional amine, a polyfunctional thiol, a surfactant, a plasticizer, a surface lubricant, a leveling agent, a softener, an antioxidant, an antiaging agent, a light stabilizer, an ultraviolet absorber, an inorganic or organic filler, and a metal powder.

[Method for Preparing Curable Composition]

A method for preparing the curable composition is not particularly limited, and a known method can be adopted. For example, the curable composition can be prepared by mixing the above-described components and then stirring the mixture by known means.

[Manufacturing Method of Sheet]

The method for manufacturing the sheet according to the embodiment of the present invention is not particularly limited, but examples thereof include a method for applying the above-described curable composition onto a base material, heating or irradiating the obtained coating film with light (as light, for example, ultraviolet rays, visible rays, electron beams, γ-rays, X-rays, and the like). That is, the cured film formed by curing the coating film can be used as an antithrombotic cell adhesion sheet.

A material of the base material is not particularly limited, and examples thereof include a glass material, a metal material, a ceramic material, and a plastic material.

Examples of a kind of the glass material include soda-lime glass, borosilicate glass, and quartz glass.

Examples of a kind of the plastic material include polyethylene terephthalate, polybutylene terephthalate, polyethylene naphthalate, polyethylene, polypropylene, cellophane, cellulose diacetate, cellulose triacetate, cellulose acetate butyrate, polyvinyl chloride, polyvinylidene chloride, polyvinyl alcohol, ethylene-vinyl acetate copolymer, polystyrene, polycarbonate, polymethylpentene, polysulfone, polyether ether ketone, polyether sulfone, polyetherimide, polyimide, fluororesin, nylon, acrylic resin, polyamide, and cycloolefin.

Examples of a kind of metal material include gold, stainless steel, cobalt-chromium alloy, amalgam alloy, silver-palladium alloy, gold-silver-palladium alloy, titanium, nickel-titanium alloy, and platinum.

Examples of a kind of the ceramic material include hydroxyapatite.

A shape of the base material is not particularly limited, and may be a plate shape or a three-dimensional shape. The base material may be a medical device to be described below.

A surface of the base material may be reformed by a surface reforming agent, plasma treatment, or the like.

A method for applying the curable composition is not particularly limited, and examples thereof include methods such as immersing, roll coating, lick roll coating, gravure coating, reverse coating, roll brushing, spray coating, dip roll coating, bar coating, spin coating, knife coating, air knife coating, curtain coating, lip coating, and extrusion coating using a die coater or the like.

A heating method is not particularly limited, and examples thereof include a method using a blast dryer, an oven, an infrared dryer, and a heating drum.

A heating temperature is not particularly limited, but is preferably 30° C. to 150° C., and more preferably 40° C. to 120° C.

A heating time is not particularly limited, but is usually 1 minute to 6 hours. In the case of drying in a coating device, a drying time is 1 minute to 20 minutes, and a heating temperature in the case of heating after coating (for example, heating in a winding form) is preferably a room temperature to 50° C.

Examples of a light irradiation method include a low-pressure mercury lamp, a medium-pressure mercury lamp, a high-pressure mercury lamp, a metal halide lamp, deep-ultraviolet (UV) light, a light emitting diode (LED) lamp, a xenon lamp, a chemical lamp, and a carbon arc lamp. Energy of the light irradiation is not particularly limited, but is preferably 0.1 to 10 J/cm².

<Sheet>

The sheet (film) formed by curing the curable composition can be used as an antithrombotic cell adhesion sheet.

A thickness of the sheet is not particularly limited, but is, for example, preferably 0.01 to 300 μm, more preferably 0.05 to 300 μm, and still more preferably 0.1 to 100 μm.

The sheet includes a polymer compound containing a repeating unit derived from the compound represented by General Formula (1) described above.

The sheet formed by curing the curable composition may be used in a state where the base material is peeled off, or may be used integrally with the base material. The base material may be a medical device to be described below.

<Cell Adhesion Properties>

In the sheet according to the embodiment of the present invention, platelets are less likely to adhere and cells are likely to adhere. That is, the sheet is suitable for a purpose of the antithrombotic cell adhesion sheet.

The sheet according to the embodiment of the present invention has excellent cell adhesion properties to normal cells and tumor cells, and in particular, has more excellent cell adhesion properties to tumor cells.

Examples of the normal cells include cells derived from tissues that maintain normal functions in epithelial tissue, connective tissue, muscle tissue, and nerve tissue.

Examples of the tumor cells include benign tumor cells and malignant tumor cells.

Examples of the malignant tumor cells include cancers such as breast cancer, fibrosarcoma, cervical cancer, prostate cancer, esophageal cancer, gastric cancer, colon cancer, pancreatic cancer, rectal cancer, gallbladder cancer, liver cancer, oropharyngeal cancer, lung cancer, and skin cancer.

[Medical Device with Sheet]

The present invention also relates to a medical device with a sheet.

Hereinafter, the medical device with a sheet will be described in detail.

The medical device with a sheet includes a medical device and an antithrombotic cell adhesion sheet (sheet according to the embodiment of the present invention) disposed on a surface of the medical device.

The sheet according to the embodiment of the present invention is as described above. The sheet according to the embodiment of the present invention can function as a coating material of the medical device.

The medical device is not particularly limited, and is preferably a medical device used for various purposes such as an antithrombotic purpose and a cell adhesion purpose.

A shape of the medical device used for the cell adhesion purpose is not particularly limited, and examples thereof include shapes such as a film, a sheet, a tube, a bag, a chalet, a dish, a well, a porous body, and a vial. The term “porous” is intended to be a material having a plurality of pores, and examples thereof include a polyimide porous film.

The medical device used for the antithrombotic purpose is not particularly limited, and examples thereof include a stent, a graft, a catheter, an artificial heart, an artificial lung, an artificial heart valve, a dilator, a vascular occluder, an embolic filter, an embolic removal device, an artificial blood vessel, a sheath, an intravascular monitoring device, a pacemaker electrode, a guide wire, a cardiac lead, a cardiopulmonary bypass circuit, a cannula, a plug, a drug delivery device, a balloon, a tissue patch device, and a blood pump. In particular, a medical device selected from the group consisting of the stent, the graft, and the catheter is preferable.

Examples of a manufacturing method of a medical device with a sheet include a method of forming a cured film (an antithrombotic cell adhesion sheet) on a surface of a medical base material by using the above-described curable composition. The method for forming the cured film using the curable composition is as described above.

The thickness of the antithrombotic cell adhesion sheet disposed on the surface of the medical base material is not particularly limited, and is, for example, preferably 0.01 to 300 μm, and more preferably 0.1 to 100 μm.

### EXAMPLES

Hereinafter, the present invention will be described in more detail based on Examples. Materials, amounts used, ratios, treatment contents, treatment procedures, and the like shown in the following examples can be appropriately changed without departing from the spirit of the present invention. Accordingly, the scope of the present invention should not be construed as being limited by the following examples.

[Manufacture and Evaluation of Cell Adhesion Sheet]

[Various Components]

Hereinafter, first, components to be used for manufacturing cell adhesion sheets of Examples and Comparative Examples will be shown.

<Polyfunctional (Meth)Acrylamide Compound>

As a polyfunctional (meth)acrylamide compound, a compound 1A to be shown below was used.

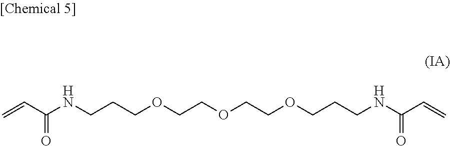 [SMILES: C=CC(=O)NCCCOCCOCCOCCCNC(=O)C=C]

<Polyfunctional Acrylate Compound>

A polyfunctional acrylate compound (compound CR to be shown below) was used as a comparative compound. As the compound CR, a commercially available product manufactured by Tokyo Chemical Industry Co., Ltd. was used.

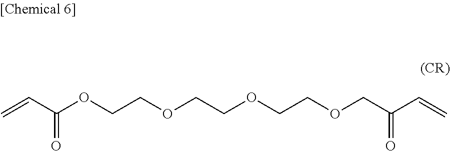 [SMILES: C=CC(=O)COCCOCCOCCOC(=O)C=C]

<Polymerization Initiator>

As the polymerization initiator, a photopolymerization initiator PI-1 to be shown below was synthesized and used.

The photopolymerization initiator PI-1 was synthesized according to the method described in paragraphs [0105] to [0110] of WO2017/018146A.

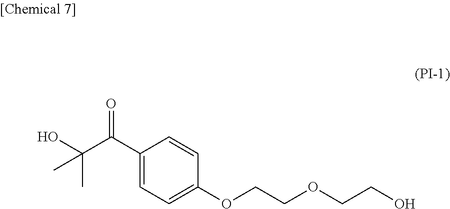 [SMILES: CC(C)(O)C(=O)c1ccc(OCCOCCO)cc1]

<Polymerization Inhibitor>

4-hydroxy-2,2,6,6-tetramethylpiperidin-1-oxyl (40H-TEMPO) was used as the polymerization inhibitor.

<Solvent>

Methanol was used as the solvent.

[Preparation and Evaluation of Cell Adhesion Sheet of Example 1]

A cell adhesion sheet of Example 1 was manufactured by a method to be shown below.

<Preparation of Curable Composition 1>

A curable composition 1 was prepared by mixing components in formulation amounts to be shown below.

| | |
|---|---|
| Polyfunctional (meth)acrylamide compound | 19.4 parts by mass |
| (compound 1A) | |
| Polymerization initiator (photopolymerization | 0.6 parts by mass |
| initiator PI-1) | |
| Polymerization inhibitor (4OH-TEMPO) | 30 mass ppm with respect |
| | to total solid content |
| Solvent (methanol) | 90.0 parts by mass |

<Preparation of Cell Adhesion Sheet>

A clearance of the prepared curable composition 1 was adjusted to a thickness of 3 μm after drying by using a bar coater, and the prepared curable composition was applied onto a polyethylene terephthalate (PET) film (COSMOSHINE A4300 manufactured by TOYOBO Co., Ltd.; easy adhesion treatment on both sides) and dried.

Thereafter, exposure was performed by using an ultraviolet exposure machine (ECS-401G manufactured by EYE GRAPHICS Co., Ltd.; light source of high-pressure mercury lamp) to have an exposure amount of 2 J/cm², and a cured film (cell adhesion sheet) was prepared on a PET film.

### Evaluation

The cell adhesion sheet of Example 1 was evaluated as follows.

<Platelet Adhesion Properties>

An adhesion experiment of platelets was performed by using the prepared cell adhesion sheet of Example 1 and a PET film (DIAFOIL T100E125 manufactured by Mitsubishi Plastics, Inc.) as a control sample. A platelet suspension of 4×10⁷ cells/mL was prepared by recovering platelet-rich blood plasma and platelet-poor blood plasma from human whole blood anticoagulated with sodium citrate by centrifugation and diluting the platelet-rich blood plasma with the platelet-poor blood plasma. Subsequently, a sample surface and the platelet suspension were brought into contact with each other at 37° C. for 60 minutes and were then rinsed twice with a phosphate buffer solution. The adhered platelets were immobilized with a 1% glutaraldehyde solution. The immobilized sample was washed by immersing the sample in the phosphate buffer solution for 10 minutes, in phosphate buffer solution:water=1:1 for 8 minutes, in water for 8 minutes, and in water for 8 minutes again, and were air-dried at a room temperature. Thereafter, the platelets adhered to the sample surface of 1×104 μm² were observed with an electron microscope, and the number of adhered platelets was measured.

A relative number of platelets adhered to the cell adhesion sheet of Example 1 in a case where a total number of platelets adhered to the PET film (control sample) was 100% was calculated, and the platelet adhesion properties were evaluated according to the following criteria. The smaller the relative number, the better the platelet adhesion properties and the better the antithrombotic properties. From the viewpoint of practicality, an evaluation of “B” or higher is preferable. The results are shown in Table 1.

A . . . 5% or less

B . . . more than 5% and 20% or less

C . . . more than 20%

<Tumor Cell Adhesion Properties>

(1) Evaluation for MCF-7 (Human-Derived Benign Breast Tumor Cell Line)

An adhesion experiment of tumor cells was performed by using, as an evaluation substrate, the prepared cell adhesion sheet of Example 1 and a PET film (DIAFOIL T100E125, manufactured by Mitsubishi Plastics, Inc.) as a control sample. A surface of the substrate was washed with phosphate-buffered saline, and then a DMEM/F12 medium (a 1:1 mixed medium of a Dulbecco's modified eagle's medium and a ham F-12 medium) prepared by adding 10% of fetal calf serum was immersed at 37° C. for 60 minutes for acclimatization. The MCF-7 (human-derived benign breast tumor cell line) suspended in the above-mentioned medium was seeded with respect to each sample at a density of 1×10⁴ cells per 1 cm² and was brought into contact with each sample at 37° C. for 60 minutes. Subsequently, the substrate was rinsed twice with a phosphate buffer solution, and the cells adhered to the substrate were fixed with a 4% paraformaldehyde solution. A nucleus of the cell was stained with DAPI (4′,6-diamidino-2-phenylindole), and an actin skeleton was stained with a phalloidin antibody. The number of adhered cells was measured by using a fluorescence microscope.

A relative number of tumor cells adhered to the cell adhesion sheet of Example 1 in a case where a total number of tumor cells adhered to the PET film (control sample) was 100% was calculated, and tumor cell adhesion properties were evaluated according to the following criteria. From the viewpoint of practicality, an evaluation of “B” or higher is preferable. The results are shown in Table 1.

A . . . more than 150%

B . . . more than 100% and 150% or less

C . . . 100%

D . . . less than 100%

(2) Evaluation of MDA-MB-231 (Human-Derived Invasive Malignant Breast Cancer Cell Line)

The tumor cell adhesion properties were evaluated by the same method as (1) the evaluation method for the MCF-7 (human-derived benign breast tumor cell line) described above except that the suspended MCF-7 (human-derived benign breast tumor cell line) was changed to the suspended MDA-MB-231 (human-derived invasive malignant breast cancer cell line).

[Preparation and Evaluation of Cell Adhesion Sheet of Comparative Example 1]

<Preparation of Curable Composition 2>

A curable composition 2 was prepared by mixing components in formulation amounts to be shown below.

| | |
|---|---|
| Polyfunctional acrylate compound (compound | 19.4 parts by mass |
| CR) | |
| Polymerization initiator (photopolymerization | 0.6 parts by mass |
| initiator PI-1) | |
| Polymerization inhibitor (4OH-TEMPO) | 30 mass ppm with respect |
| | to total solid content |
| Solvent (methanol) | 90.0 parts by mass |

<Preparation of Cell Adhesion Sheet of Comparative Example 1>

A cell adhesion sheet of Comparative Example 1 was prepared by the same method as the cell adhesion sheet of Example 1 except that the curable composition 1 was changed to the curable composition 2.

### Evaluation

Platelet adhesion properties of the obtained cell adhesion sheet of Comparative Example 1 were evaluated in the same manner as the cell adhesion sheet of Example 1. The results are shown in Table 1.

Since the evaluation of the platelet adhesion properties was poor, tumor cell adhesion properties of the cell adhesion sheet of Comparative Example 1 were not evaluated.

[Evaluation of Cell Adhesion Sheet of Comparative Example 2]

A PET film (DIAFOIL T100E125 manufactured by Mitsubishi Plastics, Inc.) was used as a cell adhesion sheet of Comparative Example 2.

The cell adhesion sheet of Comparative Example 2 was evaluated in the same manner as the cell adhesion sheet of Example 1. The results are shown in Table 1.

TABLE 1

| | Evaluation | | |
|---|---|---|---|
| | Platelet adhesion | | |
| | properties | Tumor cell adhesion properties | |
| Cell adhesion sheet | (antithrombotic) | MCF-7 | MDA-MB-231 |
| Example 1 | A | A | B |
| Comparative | C | Not performed | Not performed |
| Example 1 | | | |
| Comparative | C | C | C |
| Example 2 | | | |

In the cell adhesion sheet of Example 1, the adhesion of platelets was small and the adhesion of tumor cells was large. That is, it was clarified that the antithrombotic properties were excellent and the tumor cell adhesion properties were excellent. From the evaluation results of the tumor cell adhesion properties of Example 1, it was confirmed that the cell adhesion sheet of Example 1 exhibits good tumor cell adhesion properties to MCF-7 (human-derived benign breast tumor cell line).

On the other hand, the cell adhesion sheets of Comparative Examples did not satisfy the desired requirements.

What is claimed is:

1. An antithrombotic cell adhesion sheet formed by using a compound represented by General Formula (1),
in General Formula (1), R¹ and R⁴ each independently represent a hydrogen atom or a methyl group, R² and R³ each independently represent —CH₂CH(R^{A1})CH₂—, p and r represent 1, q represents 0 or 1, m and n each independently represent an integer of 2 to 6, and RAI represents a hydrogen atom or a methyl group.

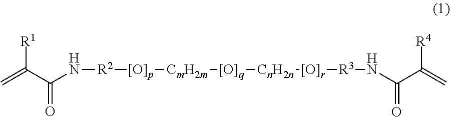

2. The antithrombotic cell adhesion sheet according to claim 1,
wherein the q represents 1.

3. The antithrombotic cell adhesion sheet according to claim 1,
wherein the R^{A1} represents a hydrogen atom.

4. The antithrombotic cell adhesion sheet according to claim 1,
wherein both the R¹ and the R⁴ represent a hydrogen atom.

5. The antithrombotic cell adhesion sheet according to claim 1,
wherein both the m and the n represent 2.

6. A medical device with a sheet comprising:
a medical device; and
the antithrombotic cell adhesion sheet according to claim 1 disposed on a surface of the medical device.

7. The medical device with a sheet according to claim 6,
wherein the medical device is selected from the group consisting of a stent, a graft, and a catheter.

8. The medical device with a sheet according to claim 6,
wherein a shape of the medical device is selected from the group consisting of a film, a sheet, a tube, a bag, a chalet, a dish, a well, a porous body, and a vial.

9. The antithrombotic cell adhesion sheet according to claim 2,
wherein the R^{A1} represents a hydrogen atom.

10. The antithrombotic cell adhesion sheet according to claim 2,
wherein both the R¹ and the R⁴ represent a hydrogen atom.

11. The antithrombotic cell adhesion sheet according to claim 2,
wherein both the m and the n represent 2.

12. A medical device with a sheet comprising:
a medical device; and
the antithrombotic cell adhesion sheet according to claim 2 disposed on a surface of the medical device.

13. The medical device with a sheet according to claim 12,
wherein the medical device is selected from the group consisting of a stent, a graft, and a catheter.

14. The medical device with a sheet according to claim 12,
wherein a shape of the medical device is selected from the group consisting of a film, a sheet, a tube, a bag, a chalet, a dish, a well, a porous body, and a vial.

15. The antithrombotic cell adhesion sheet according to claim 3,
wherein both the R¹ and the R⁴ represent a hydrogen atom.

16. The antithrombotic cell adhesion sheet according to claim 3,
wherein both the m and the n represent 2.

17. A medical device with a sheet comprising:
a medical device; and
the antithrombotic cell adhesion sheet according to claim 3 disposed on a surface of the medical device.

18. The medical device with a sheet according to claim 17,
wherein the medical device is selected from the group consisting of a stent, a graft, and a catheter.

19. The medical device with a sheet according to claim 17,
wherein a shape of the medical device is selected from the group consisting of a film, a sheet, a tube, a bag, a chalet, a dish, a well, a porous body, and a vial.

20. The antithrombotic cell adhesion sheet according to claim 4,
wherein both the m and the n represent 2.
